(19) United States
(12) Patent Application Publication
(10) Pub. No.: US 2017/0296221 A1
(43) Pub. Date: Oct. 19, 2017

(54) Catheter for Advancing Through a Vascular Stenosis and Related Methods

(71) Applicant: QXMedical, LLC., Roseville, MN (US)

(72) Inventors: Fernando Di Caprio, St. Paul, MN (US); Gianfranco Panarello, Montreal (CA)

(21) Appl. No.: 15/490,200

(22) Filed: Apr. 18, 2017

Related U.S. Application Data
(60) Provisional application No. 62/324,029, filed on Apr. 18, 2016.

(51) Int. Cl. A61B 17/3207; A61M 25/00; A61M 25/01; A61M 25/09; A61M 25/10; A61B 17/32

(52) U.S. Cl. CPC A61B 17/320758; A61M 25/0105; A61M 25/0045; A61M 25/005; A61M 25/0068; A61M 25/01; A61M 25/10; A61M 25/09; A61B 17/32075; A61B 2017/320008; A61M 2025/0046; A61M 2025/006; A61M 2025/0183; A61M 2025/0188

(57) ABSTRACT

The various embodiments disclosed herein relate to catheters for advancing past a vascular stenosis. Each of these catheters has at least one friction-reducing feature to reduce the friction between the catheter and the stenosis as the catheter is urged therethrough.

### CROSS-REFERENCE TO RELATED APPLICATION(S)

This application claims the benefit of U.S. Provisional Application 62/324,029, filed on Apr. 18, 2016 and entitled “Catheter for Advancing Through a Vascular Stenosis and Related Methods,” which is hereby incorporated herein by reference in its entirety.

### FIELD OF THE INVENTION

The various embodiments disclosed herein relate to catheters for use in cardiovascular procedures, and more specifically to catheters configured to be advanced past a narrowed portion of a blood vessel.

### BACKGROUND OF THE INVENTION

During interventional cardiology procedures, a catheter often must be advanced beyond a narrowing vascular lesion, or vascular stenosis, in a blood vessel to advance to a target area. While it is often possible to pass a guidewire through a blockage or narrow passage created by such a stenosis, it is often difficult or impossible to advance a larger device, such as a balloon or stent carried by an intravascular catheter, through that blockage. A typical catheter may have excessive frictional forces which prevent it from crossing the constriction.

There is a need in the art for an improved catheter for advancing past narrowed, blocked, or restricted portions of a blood vessel.

### BRIEF SUMMARY OF THE INVENTION

Discussed herein are various catheters and related methods configured to advance past or through a vascular stenosis.

In Example 1, a catheter for advancing past a vascular stenosis comprises a catheter body comprising a lumen defined therein, and at least one friction-reducing feature associated with the catheter body.

Example 2 relates to the catheter according to Example 1, wherein the at least one friction reducing feature is disposed on an outer surface of the catheter body.

Example 3 relates to the catheter according to Example 1, further comprising a tip coupled to a distal end of the catheter body, wherein the at least one friction reducing feature is disposed on an outer surface of the tip.

Example 4 relates to the catheter according to Example 1, wherein the at least one friction-reducing feature comprises a plurality of projections.

Example 5 relates to the catheter according to Example 4, wherein the plurality of projections comprise bumps, quadrangular projections, or triangular projections.

Example 6 relates to the catheter according to Example 1, wherein the at least one friction-reducing feature comprises a plurality of openings or dimples defined in the outer surface of the catheter body.

Example 7 relates to the catheter according to Example 1, wherein the at least one friction-reducing feature comprises a plurality of grooves, nubs, ribs, or textured features.

Example 8 relates to the catheter according to Example 1, wherein the at least one friction-reducing feature comprises at least one wire formed into a coil configuration or a braided configuration.

Example 9 relates to the catheter according to Example 1, wherein the at least one friction-reducing feature comprises at least one offset projection.

Example 10 relates to the catheter according to Example 1, further comprising an exterior layer disposed over an outer surface of the catheter body and the at least one friction-reducing feature.

Example 11 relates to the catheter according to Example 1, further comprising a lubricious coating disposed over at least an outer surface of the catheter body.

Example 12 relates to the catheter according to Example 1, wherein the catheter body comprises a length having a reduced diameter, wherein the at least one friction reducing feature is disposed on an outer surface of the length having the reduced diameter.

Example 13 relates to the catheter according to Example 12, further comprising a tip positioned over the length having the reduced diameter, wherein the at least one friction reducing feature is disposed on an outer surface of the tip.

Example 14 relates to the catheter according to Example 1, wherein the catheter shaft is a hypotube.

Example 15 relates to the catheter according to Example 14, wherein the catheter shaft further comprises at least two slots defined in the a distal end of the catheter shaft.

Example 16 relates to the catheter according to Example 15, wherein the at least two slots comprise a straight configuration or a spiral-like configuration.

Example 17 relates to the catheter according to Example 1, wherein the catheter shaft comprises a first length comprising a first diameter, a second length comprising a second diameter, and a transition portion disposed between the first and second lengths.

Example 18 relates to the catheter according to Example 1, wherein the catheter shaft comprises a first layer and a second layer.

Example 19 relates to the catheter according to Example 1, wherein the catheter comprises an over-the-wire catheter or a rapid-exchange catheter.

Example 20 relates to the catheter according to Example 1, further comprising a rotation mechanism associated with a distal end of the catheter body, wherein the rotation mechanism is configured to rotate when actuated.

In Example 21, a method of advancing a catheter past a vascular stenosis comprises positioning a catheter into a blood vessel and advancing the catheter past the vascular stenosis. The catheter comprises a catheter body comprising a lumen defined therein and at least one friction-reducing feature associated with the catheter body. The at least one friction reducing feature reduces friction between the vascular stenosis and the catheter.

Example 22 relates to the method according to Example 21, further comprising rotating a distal end of the catheter body, wherein the rotation reduces friction between the vascular stenosis and the catheter.

Example 23 relates to the method according to Example 21, further comprising moving the distal end of the catheter body in a lateral direction, wherein the movement reduces friction between the vascular stenosis and the catheter.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side view of a catheter for advancing past a vascular stenosis, according to one embodiment.

FIG. 2A is a side view of friction-reducing features on a catheter, according to one embodiment.

FIG. 2B is a side view of friction-reducing features on a catheter, according to another embodiment.

FIG. 2C is a side view of a tip having friction-reducing features on a catheter, according to one embodiment.

FIG. 2D is a side view of friction-reducing features on a catheter, according to a further embodiment.

FIG. 2E is a side view of friction-reducing features on a catheter, according to yet another embodiment.

FIG. 2F is a side view of friction-reducing features on a catheter, according to another embodiment.

FIG. 2G is a side view of friction-reducing features on a catheter, according to a further embodiment.

FIG. 2H is a side view of friction-reducing features on a catheter, according to yet another embodiment.

FIG. 2I is a side view of friction-reducing features on a catheter, according to another embodiment.

FIG. 2J is a side view of a tip having friction-reducing features on a catheter, according to another embodiment.

FIG. 2K is a side view of a tip having friction-reducing features on a catheter, according to a further embodiment.

FIG. 2L is a side view of a catheter with a neck and a tip having friction-reducing features, according to one embodiment.

FIG. 2M is a side view of a catheter with a neck and friction-reducing features, according to another embodiment.

FIG. 2N is a side view of a catheter having a friction-reducing feature, according to another embodiment.

FIG. 2O is a front view of the catheter of FIG. 2N.

FIG. 2P is a side view of a catheter having a friction-reducing feature, according to a further embodiment.

FIG. 2Q is a front view of the catheter of FIG. 2P.

FIG. 2R is a side view of a catheter having a friction-reducing feature, according to yet another embodiment.

FIG. 2S is a front view of the catheter of FIG. 2R.

FIG. 2T is a side view of a catheter having a neck and a friction-reducing feature, according to a further embodiment.

FIG. 2U is a front view of the catheter of FIG. 2T.

FIG. 2V is a side view of a catheter having friction-reducing features and an external layer, according to one embodiment.

FIG. 3 depicts a catheter having friction-reducing features being urged past or through a vascular stenosis, according to one embodiment.

FIG. 4 depicts a standard catheter being attempted to be advanced past or through a vascular stenosis.

FIG. 5 depicts a catheter having friction-reducing features and a rotating distal portion being urged past or through a vascular stenosis, according to one embodiment.

FIG. 6 is a side view of an over-the-wire catheter having friction-reducing features, according to one embodiment.

FIG. 7 is a side view of a catheter having a metal shaft and a polymeric tube with friction-reducing features, according to one embodiment.

FIG. 8A is a side view of an over-the-wire catheter having a metal shaft, slots, and friction-reducing features, according to one embodiment.

FIG. 8B is a side view of an over-the-wire catheter having a metal shaft, slots, and friction-reducing features, according to another embodiment in which the shaft has a smaller diameter than the catheter of FIG. 8A.

FIG. 9A is a side view of a rapid-exchange catheter having a shaft, a polymeric tube, and friction-reducing features, according to one embodiment.

FIG. 9B is an expanded side view of the catheter of FIG. 9A.

FIG. 10A is a side view of a rapid-exchange catheter having a metal shaft, a polymeric covering, and friction-reducing features, according to one embodiment.

FIG. 10B is an expanded side view of the catheter of FIG. 10A.

FIG. 11A is a side view of catheter having a metal shaft, slots, and friction-reducing features, according to another embodiment.

FIG. 11B is an expanded view of slots on a catheter, according to one embodiment.

FIG. 11C is an expanded view of slots on a catheter, according to another embodiment.

FIG. 11D is a side view of a polymeric tube having friction-reducing features that can be positioned through the catheter of FIG. 11A, according to one embodiment.

### DETAILED DESCRIPTION

The various embodiments disclosed herein relate to catheters that can be advanced past or through a narrowed or blocked portion of a blood vessel. More specifically, certain catheter implementations have friction-reducing features situated along at least a portion of the outer surface of the catheter. Alternative embodiments utilize rotational or torsional motion along the length of the device disposed within the blood vessel to further reduce friction.

In the various embodiments herein, the catheter is advanced past the narrowed portion of the blood vessel, thereby creating, enhancing, or enlarging a passageway for the subsequent advancement of one or more additional intravascular devices or catheters (such as, for example, a guidewire, a balloon catheter, a stent delivery system, a support catheter, etc.). The passageway can be the result of physically enlarging or expanding the diameter of the blood vessel at the narrowed portion as a result of advancing the catheter past that portion (a process commonly referred to as “dottering”). Further, after advancement of the catheter past the narrowed portion, the various catheter implementations herein can be used to exchange guidewires. That is, the initial guidewire that is positioned through the blood vessel for insertion of the catheter embodiments contemplated herein can be relatively compliant in comparison to other guidewires such that the guidewire can be advanced past the restriction but does not provide sufficient support for advancement of an interventional device. In such scenarios, the various catheter embodiments disclosed or contemplated herein can be used to exchange the compliant guidewire for a more robust wire such that an interventional device can then be advanced past the restriction.

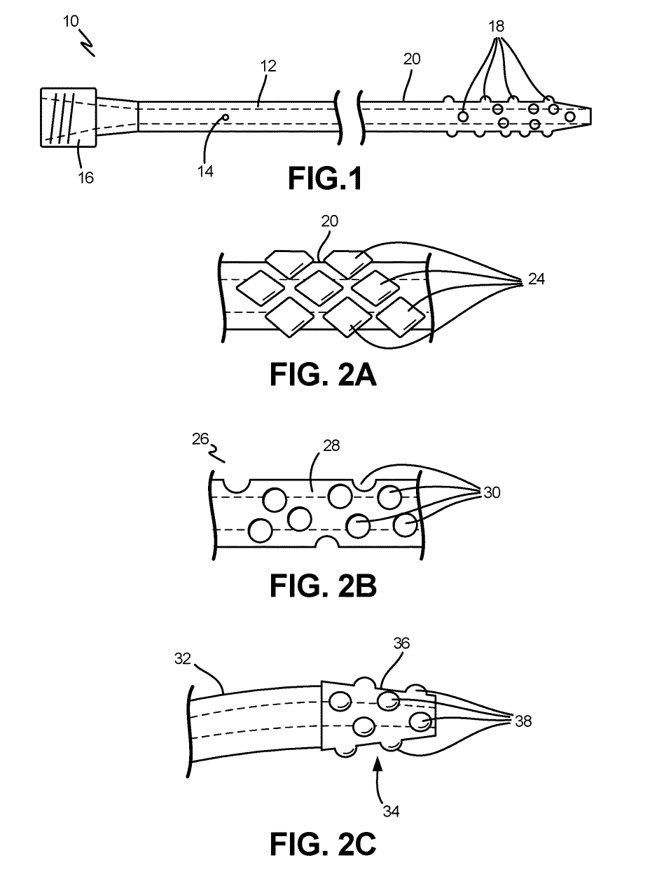

One embodiment of a catheter 10 with friction-reducing features is depicted in FIG. 1. The catheter 10 has a catheter body 12, a lumen 14 defined within the body 12 for passage of a guidewire, procedure fluids, etc., and a handle 16. In addition, the catheter 10 also has a plurality of surface features 18 along the outer surface 20 of a distal portion of the body 12. In this exemplary implementation as shown, the surface features 18 are bumps, nubs, or projections 18 that extend away from the outer surface 20 of the body 12.

According to one embodiment, during use, the surface features 18, and any other surface feature embodiments disclosed or contemplated herein, reduce the contact area between the catheter 10 and the inner walls of the vessel in which it is positioned. The reduced contact area will result in lower frictional forces and thus decrease the force required to urge the catheter through or past a constriction.

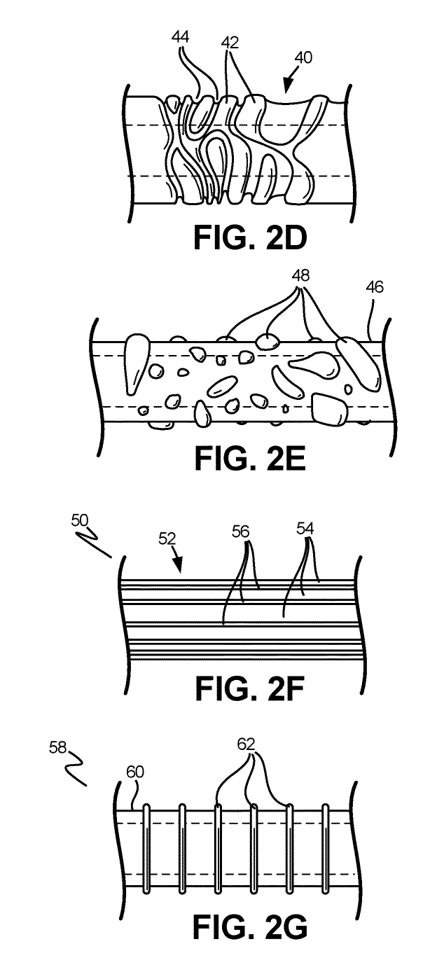

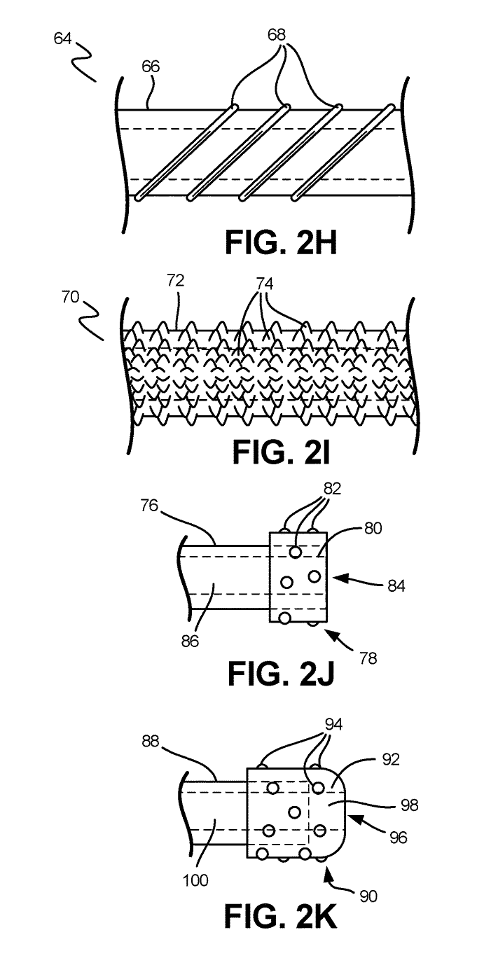

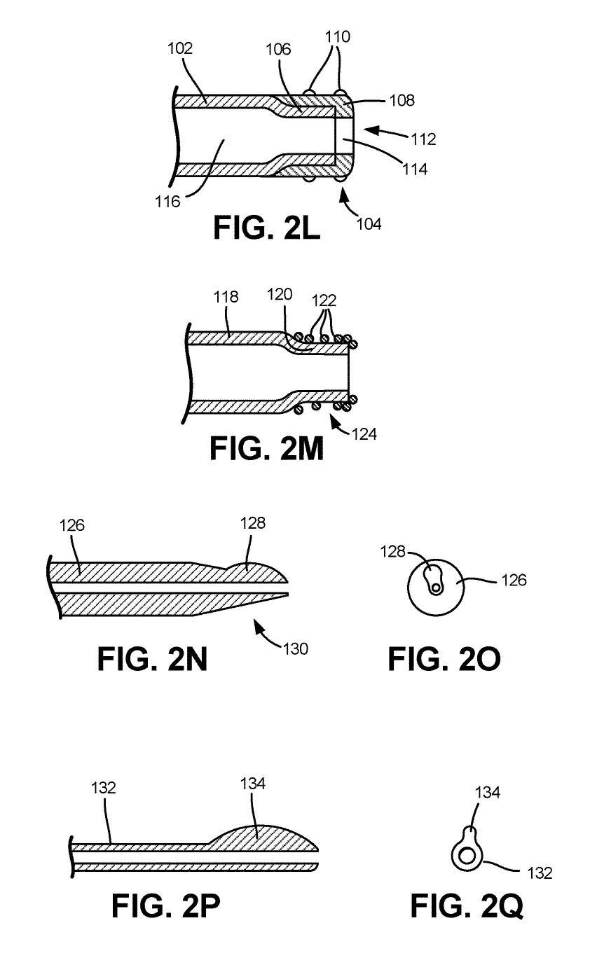

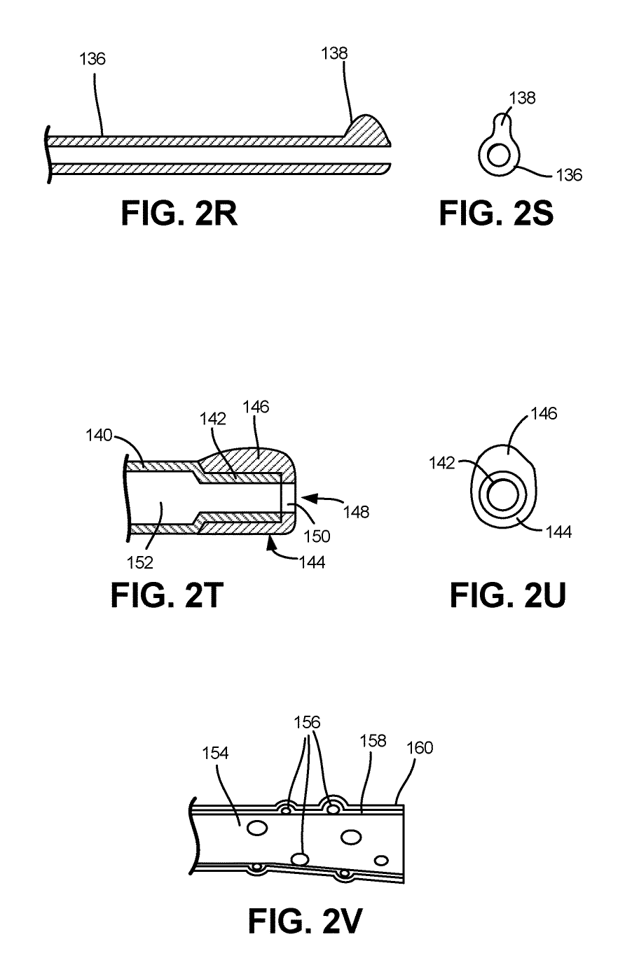

Another embodiment of friction-reducing surface features 24 is depicted in FIG. 2A, in which the surface features 24 are shaped projections 24 disposed on the outer surface 20. More specifically, the shaped projections 24 include both quadrangular and triangular projections 24 as shown. Alternatively, it is understood that the projections can take any geometrical shape.

FIG. 2B depicts another implementation of friction-reducing surface features 30. In this particular embodiment, the features 30 are openings or holes 30 defined in the catheter body 12. Alternatively, the features 30 can be divets or dimples 30, which are opening-like features that do not extend all the way through the catheter body 12. Like protrusions or projections (such as the projections 18, 24 discussed above) or other types of features, these openings 30 reduce friction by reducing the contact area between the catheter 26 and the vessel inner walls.

A further embodiment is depicted in FIG. 2C, relating to a tip 34 that is attachable to or positionable on the distal end of a catheter shaft 32. In one embodiment, the tip 34 is permanently positioned on the distal end of the shaft 32. Alternatively, the tip 34 is removable. The tip 34 has a plurality of surface features 38 along the outer surface 36 of the tip 34. In this exemplary implementation as shown, the surface features 38 are projections 38. Alternatively, the features 38 can be any known friction-reducing features as described or contemplated herein. According to one implementation, the tip 34 is made of metal. Alternatively, the tip 34 can be made of any known material for use in a catheter.

Yet another implementation is shown in FIG. 2D, in which the friction-reducing surface features 42 are defined by grooves 44 that are formed in the outer surface 40. In one embodiment, the grooves 44 are formed using a laser cutting process. Alternatively, the grooves 44 can be formed in any known fashion.

Another embodiment is depicted in FIG. 2E, which shows friction-reducing surface features 48 that are pebbles or nubs 48 formed on the outer surface 46. In one embodiment, the nubs 48 are formed by adding a polymeric material to the outer surface 46 in pebble-like configurations or shapes as shown. Alternatively, the nubs 48 are formed by ablating away or otherwise removing material from the outer surface 46 such that the nubs 48 are the remaining, unremoved material.

A further implementation of friction-reducing surface features 54 is depicted in FIG. 2F, in which the surface features 54 are axially-oriented or axially-disposed ribs 54 running along the length of the outer surface 52 of the catheter 50. In one embodiment, the ribs 54 are formed by adding a material to the outer surface 52 to form the ribs 54 as shown. Alternatively, the ribs 54 are formed by ablating away or otherwise removing material from the outer surface 52 to form grooves 56 such that the ribs 54 are the remaining, unremoved material.

Yet another embodiment is shown in FIG. 2G, in which the friction-reducing surface features 62 are circumferentially-oriented or transversely-disposed ribs 62 disposed around the circumference of the outer surface 60 of the catheter 58. In one embodiment, the ribs 62 are formed by adding a material to the outer surface 60 to form the ribs 62 as shown. Alternatively, the ribs 62 are formed by ablating away or otherwise removing material from the outer surface 60 such that the ribs 62 are the remaining, unremoved material.

Another implementation is depicted in FIG. 2H, which shows friction-reducing surface features 68 that are angled circumferentially-oriented or transversely-disposed ribs 68 disposed around the circumference of the outer surface 66 of the catheter 64. In one embodiment, the ribs 68 are formed by adding a material to the outer surface 66 to form the ribs 68 as shown. Alternatively, the ribs 68 are formed by ablating away or otherwise removing material from the outer surface 66 such that the ribs 68 are the remaining, unremoved material.

A further embodiment of friction-reducing surface features 74 is depicted in FIG. 2I, in which the surface features 74 are textured surface features 74 that are created by abrading the outer surface 72 of the catheter 70. In one implementation, the textured surface features 74 have a micro-pebble pattern 74. According to one embodiment, the texturing process can be accomplished by a mechanical, chemical, or electrical abrasion process. Alternatively, the textured surface features 74 are formed by depositing the features 74 onto the outer surface 72 using a known deposition process.

According to other implementations similar to FIG. 2C (discussed above), a tip is added to the distal end of the catheter. One such exemplary embodiment is depicted in FIG. 2J, in which the catheter shaft 76 has a tip 78 that is attachable to or positionable on the distal end of the shaft 76. In one embodiment, the tip 78 is permanently positioned on the distal end of the shaft 76. Alternatively, the tip 78 is removable. The tip 78 has a plurality of surface features 82 along the outer surface 80 of the tip 78. In this exemplary implementation as shown, the surface features 82 are projections or nubs 82. Alternatively, the features 82 can be any known friction-reducing features as described or contemplated herein. According to one implementation, the tip 78 is made of a polymeric material and the shaft 76 is metal. Alternatively, the tip 78 can be made of any known material for use in a catheter and the shaft 76 can be made of any known material for a shaft.

In this specific implementation, the tip 78 is positioned at the end of the shaft 76 such that the distal end of the tip 78 ends at the distal end of the shaft 76. In other words, the ends of the tip 78 and the shaft 76 are flush—the distal end of the tip 78 does not extend beyond the distal end of the shaft 76. An opening 84 at the distal end of the tip 78 and shaft 76 is in fluidic communication with an inner lumen 86 of the shaft 76.

FIG. 2K depicts an alternative embodiment of a catheter shaft 88 having a distal tip 90. In this implementation, the tip 90 is permanently positioned on the distal end of the shaft 88. Alternatively, the tip 90 is removable. The tip 90 has a plurality of surface features 94 that are projections or nubs 94 along the outer surface 92 of the tip 90. Alternatively, the features 94 can be any known friction-reducing features as described or contemplated herein. According to one implementation, the tip 90 is made of a polymeric material and the shaft 88 is metal. Alternatively, the tip 90 can be made of any known material for use in a catheter and the shaft 88 can be made of any known material for a shaft. In this specific implementation, the tip 90 is positioned at the end of the shaft 88 such that the distal end of the tip 90 extends past the distal end of the shaft 88. An opening 96 at the distal end of the tip 90 is in fluidic communication with an inner lumen 98 of the tip 90 that is in fluidic communication with the inner lumen 100 of the shaft 88.

A further implementation of a catheter shaft 102 with a distal tip 104 is shown in FIG. 2L. In this implementation, the shaft 102 has a distal end with a length 106 having a reduced diameter (also referred to herein as a “neck”) such that the tip 104 is positioned over the neck 106 in such a fashion that the outer diameter of the tip 104 is substantially the same as the outer diameter of the shaft 102 proximal to the neck 106. Alternatively, the tip 104 can have an outer diameter that is larger or smaller than the outer diameter of the shaft 102. In one embodiment, the tip 104 is permanently positioned on the distal end of the shaft 102. Alternatively, the tip 104 is removable. The tip 104 has a plurality of surface features 110 that are projections or nubs 110 along the outer surface 108 of the tip 104. Alternatively, the features 110 can be any known friction-reducing features as described or contemplated herein. According to one implementation, the tip 104 is made of a polymeric material and the shaft 102 is metal. Alternatively, the tip 104 can be made of any known material for use in a catheter and the shaft 102 can be made of any known material for a shaft. In this specific implementation, the tip 104 is positioned at the end of the shaft 102 such that the distal end of the tip 104 extends past the distal end of the shaft 102. An opening 112 at the distal end of the tip 104 is in fluidic communication with an inner lumen 114 of the tip 104 that is in fluidic communication with the inner lumen 116 of the shaft 102.

FIG. 2M depicts yet another embodiment in which the catheter shaft 118 has a neck 120 and the friction-reducing features 122 disposed on the neck 120 are the coils 122 of a coil wrap 124. Alternatively, the catheter shaft 118 has no neck 120 and the coils 122 are disposed around the distal end of the shaft 118 without a neck. In one implementation, the coils 122 are positioned randomly along the neck 120 (or distal end of the shaft 118 without a neck) such that there are gaps between the coils 122. Alternatively, the coils 122 are positioned in a predetermined, uniform pattern, such as having the coils in a tightly wound (or fully touching) configuration. In a further alternative, the features 122 can be any known friction-reducing features as described or contemplated herein. According to one implementation, the coils 122 are made of a metallic material and the shaft 118 is polymeric. Alternatively, the coils 122 can be made of any known material for use in a catheter and the shaft 118 can be made of any known material for a shaft, including metal. In accordance with certain embodiments, the coils 122 can be made of a single wire or filament or alternatively can be made of multiple wires or filaments arranged in a braided configuration, such as a hollow braid configuration. According to some implementations, the coils 122 (in any coiled or braided configuration) can extend over any length of the catheter shaft 118. Alternatively, the coils 122 can extend past the distal end of the catheter shaft 118 and be folded inward into the inner lumen of the shaft 118 in an invaginating manner (not shown).

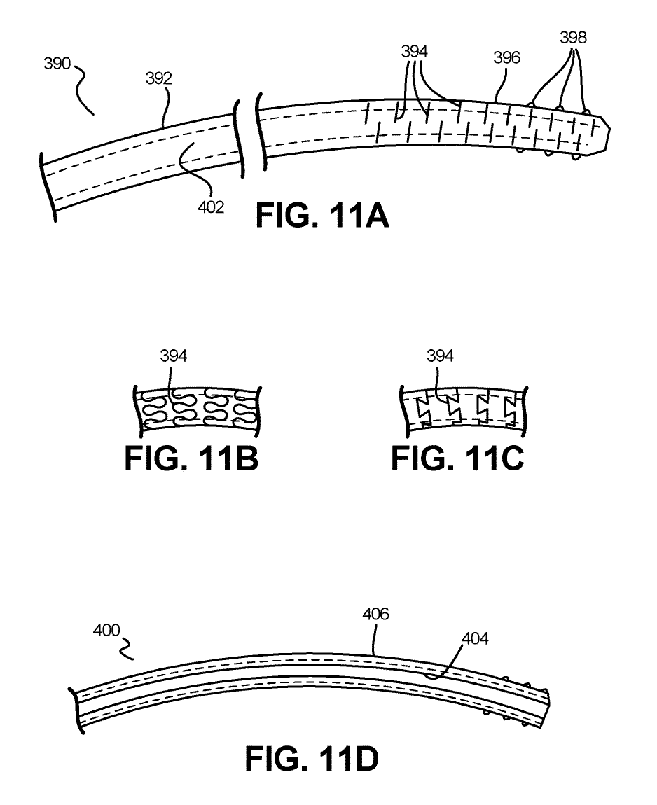

According to one implementation, any tip component disclosed or contemplated herein—such as, for example, tips 34, 78, 90, 104 discussed above and tip 144 discussed below—can be configured to extend past the distal end of the catheter shaft on which it is positioned and have an inward fold at its distal end such that the folded portion is disposed within the inner lumen of that shaft in an invaginating manner. Alternatively, any inner lumen liner (such as, for example, the inner lumen liner 400 depicted in FIG. 11D and discussed below) can be configured to extend past the distal end of the catheter shaft in which it is disposed and have an outward fold at its distal end such that the folded portion is disposed on an outer surface of the shaft.

Further friction-reducing feature embodiments are disclosed in FIGS. 2N-2U, in which the features relate to single features that are offset or asymmetrical components as described below. For example, FIGS. 2N and 2O depict a catheter shaft 126 having a single friction-reducing offset projection 128 at the distal end 130 of the shaft 126. In this implementation, the projection 128 is positioned along a length of the distal end 130 of the shaft 126 that is an angled distal end 130.

FIGS. 2P and 2Q depict another embodiment of a catheter shaft 132 having a single friction-reducing offset projection 134 at the distal end of the shaft 132. In this implementation, the shaft 132 has a relatively uniform outer diameter (in comparison to the angled distal end of the shaft 126 discussed above) such that the projection 134 creates a larger total diameter of the shaft 132 at the projection 134 as shown.

FIGS. 2R and 2S show yet another implementation of a catheter shaft 136 having a single friction-reducing offset projection 138 at the distal end of the shaft 136. As in the embodiment of FIGS. 2P and 2Q, this implementation has a shaft with a relatively uniform outer diameter such that the projection 138 creates a larger total diameter of the shaft 136 at the projection 138 as shown. In this implementation, the projection 138 is shorter in axial length in comparison to the projection 134 discussed above.

In a further implementation, a catheter shaft 140 is shown in FIGS. 2T and 2U having a distal tip 144 with a single friction-reducing offset projection 146. In this implementation, the shaft 140 has a neck 142 such that the tip 144 is positioned over and attached to the neck 142. In one embodiment, the tip 144 is permanently positioned on the distal end of the shaft 140. Alternatively, the tip 144 is removable. The tip 144 in this implementation has a diameter that is substantially similar to the outer diameter of the shaft 140 except for the projection 146, such that the projection 146 creates a larger total diameter of the tip 144 at the projection 146 as shown. Alternatively, the tip 144 can have a diameter that is larger than or smaller than the outer diameter of the shaft 140. In this specific implementation, the tip 144 is positioned at the end of the shaft 140 such that the distal end of the tip 144 extends past the distal end of the shaft 140. An opening 148 at the distal end of the tip 144 is in fluidic communication with an inner lumen 150 of the tip 144 that is in fluidic communication with the inner lumen 152 of the shaft 140.

FIG. 2V depicts a further embodiment of a catheter shaft 154 having friction-reducing features 156. In this implementation, the features 156 are beads 156 disposed on the outer surface 158 of the shaft 154 with an exterior layer 160 positioned over the outer surface 158 and beads 156. Alternatively, the features 156 can be any of the features disclosed or contemplated herein. According to one embodiment, the exterior layer 160 is a heat-shrink layer 160 of known heat-shrink material. It is understood that according to various alternative embodiments, an exterior layer 160 can be positioned over any of the various friction reducing features disclosed or contemplated herein, including the coils discussed above.

In the various embodiments disclosed above in FIGS. 1-2V and disclosed or contemplated elsewhere herein, the friction-reducing features are distributed in a random pattern across the outer surface of the catheter. Alternatively, the features can be distributed in a uniform, organized, or predetermined pattern. In a further alternative, the features do not form a threaded or helical configuration. In other words, in certain embodiments, the features can have a non-threaded or non-helical configuration. In some implementations, the friction-reducing features can form a threaded or helical configuration that is not configured to assist with advancement of the catheter past a narrowed portion of a blood vessel.

It is understood that any of the embodiments herein, including the catheter shafts and the various friction-reducing features and distal tips, can be made of any material. Any of the components can be made of metal, polymeric material, or any other known material for use in catheters. It is also understood that any of the various implementations disclosed or contemplated herein can have a lubricious coating disposed over all or some of the various components disclosed or contemplated herein, including, for example, any of the friction-reducing features and/or the layer discussed above. According to one implementation, the coating can be either hydrophilic or hydrophobic. In certain examples, the coating can be a hydrophilic coating made of polyvinyl alcohol (“PVA”), polyvinylpyrrolidone, any other known hydrophilic material, or any combination thereof. In other examples, the coating can be a hydrophobic coating made of silicone, oil, any other known hydrophobic material, or any combination thereof.

It is further understood that any of the friction-reducing features described or contemplated herein can be incorporated into any appropriate catheter for use in advancing the catheter through any narrowed portion of a blood vessel, including a vascular stenosis. For example, the catheter could be a catheter having multiple outer diameters along its length. For example, the catheter could have three lengths having different diameters and two transition portions therebetween. Alternatively, the catheter can have only two portions or lengths of uniform diameter and only one transition portion. In further alternatives, the catheter can have four or more portions or lengths of uniform diameter and three or more transition portions.

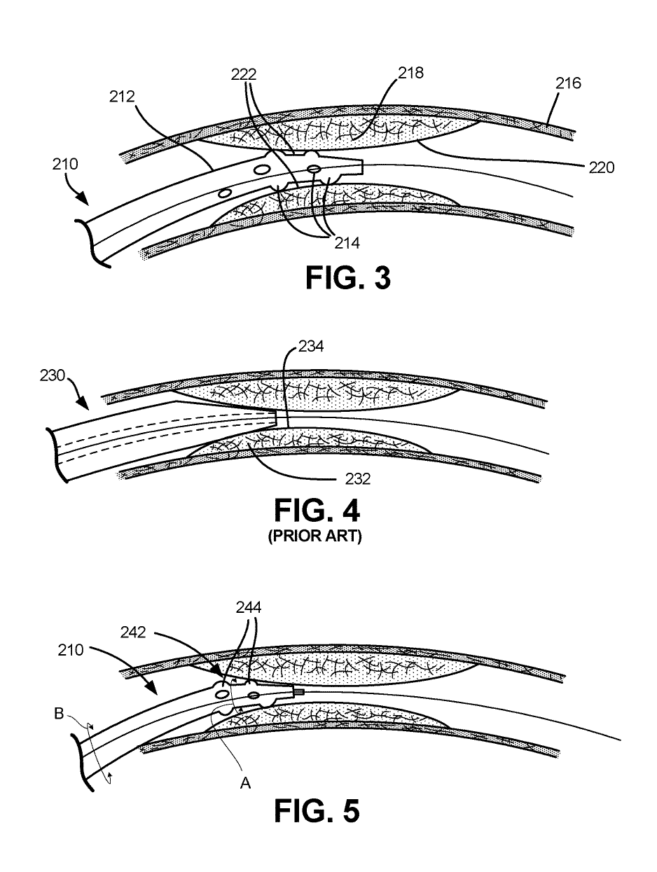

In use, according to one embodiment as shown in FIG. 3, a catheter 210 with friction-reducing features 214 is advanced through a vascular stenosis 218 of a blood vessel 216. The features 214—which in this specific implementation are projections 214—decrease the contact area between the outer surface 212 of the catheter 210 and the surface 220 of the stenosis 218 (note the gaps 222 between the outer surface 212 of the catheter 210 and the surface 220 of the stenosis 218 created by the projections 214). This reduced contact area reduces the amount of friction between the catheter 210 and the surface 220 and makes it easier for a user to advance the catheter 210 through the stenosis 218.

In contrast, a standard catheter with no friction-reducing features is shown in FIG. 4. Note that the absence of the friction-reducing features on the catheter 230 results in comparatively greater contact area (by comparison to the catheter embodiment with friction-reducing features in FIG. 3) between the device 230 and the surface 234 of the stenosis 232, thereby making it relatively more difficult to advance the device 230 through or past the stenosis 232.

According to another embodiment, certain catheters contemplated herein are configured to convert static friction to dynamic friction by utilizing rotational or torsional motion at the distal end or along a distal portion of the catheter shaft. For example, one embodiment depicted in FIG. 5 shows a catheter 240 having a rotating distal portion 242 (with the rotation represented by the arrow identified with reference letter A). In this specific example, the catheter 240 also has friction-reducing features 244. Certain alternative embodiments with a rotating distal portion have no friction-reducing features as described or contemplated herein.

In one embodiment, the rotating distal portion 242 results from the catheter 240 being configured to allow for rotation by a user at the proximal end of the catheter 240 (such as rotation of the handle (not shown), for example) such that the torque or rotation is transmitted along the length of the catheter shaft 246 (as shown by the arrow identified with reference letter B) such that the distal portion 242 rotates as shown by arrow A. In this embodiment, the catheter shaft 246 must be constructed to allow for such transmission of torque or rotation. For example, in one example, the shaft 246 is made of multiple layers of material, which could, in certain embodiments, include metal coils. Other layers might include braids, wire constructions, laser-cut hypotubes, certain polymers, or any other known layers or configurations that can provide the strength to allow for transmission of rotation or torque along the length of the shaft 246.

In certain embodiments, the rotation at the distal portion 242 is a continuous rotation. Alternatively, the rotation is a continuously repeated back-and-forth or oscillating rotation such that the distal portion 242 rotates first in one direction and then in the other direction. This back-and-forth rotation can be accomplished manually (by a user rolling the handle of the catheter 240 back and forth in her fingers), or by some mechanism or component in the catheter 240. For example, the catheter 240 could have a rotation mechanism or component (not shown) on the distal portion 242 of the catheter 240, with the rotation mechanism having a motor (not shown) disposed in or on the catheter 240 (such as at the proximal end thereof) and coupled to the mechanism such that the motor (not shown) can be actuated by a user to cause the rotation mechanism (not shown) to rotate back and forth. Alternatively, the catheter 240 can have a rotation mechanism (not shown) with a spring or other tensioned device (not shown) that causes the rotation mechanism (not shown) at the distal portion 242 to rotate back and forth. For example, in one embodiment, the rotation mechanism (not shown) with the spring device can operate in the same fashion as a wind-up device such as a “wind-up toy,” in which the device can be wound by a user and then released.

For any of the catheter embodiments disclosed or contemplated herein, the catheter shaft can be constructed in any known manner. For example, the shaft can be a single lumen configuration with a single material. Alternatively, the shaft can be a single lumen configuration with multiple layers of different materials and designs (such as coils, polymers, braids, etc.).

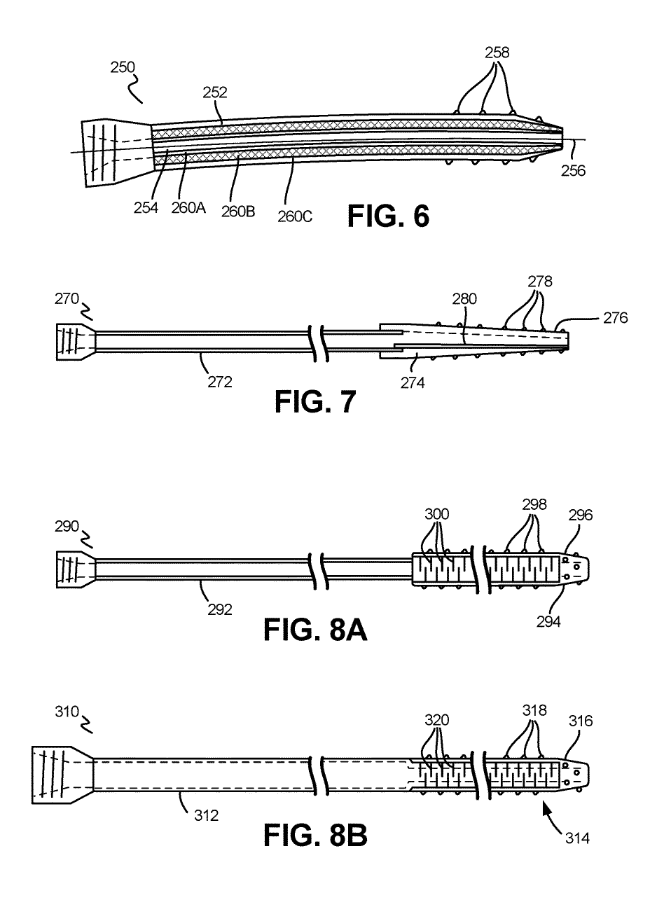

In certain implementations, the catheter embodiments disclosed or contemplated herein are over-the-wire (“OTW”) catheters. One such exemplary embodiment is depicted in FIG. 6, which depicts a catheter 250 having a shaft 252 with a lumen 254 defined therein that is configured to receive a guidewire 256 such that the guidewire 256 can be positioned through the entire length of the catheter 250. In this embodiment, the friction-reducing features 258 are beads or nubs 258 as shown.

In one implementation, the catheter 250 has a layered configuration, with an inner layer 260A, a middle layer 260B, and an outer layer 260C. The layers 260A, 260B, 260C can be made of different materials. For example, one or more of the layers 260A, 260B, 260C can be made of metal while one or more of the layers 260A, 260B, 260C can be made of a polymeric material.

Another embodiment of an OTW catheter 270 is depicted in FIG. 7, in which the catheter 270 has a metal shaft 272 (also referred to as a hypotube 272) with a polymeric tube 274 coupled to the distal end of the shaft 272. In some embodiments, the tube 274 is a solid tube 274, while in other embodiments, the tube 274 is multi-layered. The tube 274 in this embodiment has friction-reducing features 278 disposed on the outer surface 276 of the tube 274. In certain implementations, the tube 274 is tapered as shown. According to some embodiments, the tube 274 has a support rod or wire 280 coupled to the hypotube 272 and disposed within the tube 274 such that the support rod 280 provides additional structural support and/or stiffness to the tube 274.

In some embodiments, the shaft 272 has an external coating (not shown) that can be made of polymeric material such as PTFE to reduce friction. Alternatively, any known friction-reducing coating can be used.

Yet another implementation of an OTW catheter is shown in FIG. 8A, in which the catheter 290 has a metal shaft 292 (also referred to as a hypotube 292) with a polymeric covering 294 disposed on the distal end of the shaft 292. The polymeric covering 294 has friction-reducing features 298 disposed on the outer surface 296 of the covering 294. According to certain embodiments, the distal end of the shaft 292 also has slots (also referred to as “slits,” “openings,” “spaces,” or “gaps”) 300 defined in the distal end that provide additional flexibility or reduced stiffness to the distal end of the shaft 292. The slots 300 can be substantially straight slots 300 as shown. Alternatively, the slots 300 can be configured in a partially or substantially spiral-like configuration. In a further alternative, the slots 300 can have on any shape or configuration of an opening that can provide additional flexibility or reduced stiffness to the shaft 292. In accordance with one embodiment, the slots 300 can be configured such that the hypotube 292 is more flexible in the distal portion of the slots 300 in comparison to the proximal portion of the slots 300. That is, the hypotube 292 exhibits increased flexibility in a distal direction along the slots 300.

FIG. 8B depicts a further embodiment of an OTW catheter 310 having a metal shaft (“hypotube”) 312 with a polymeric covering 314 disposed on the distal end of the shaft 312, wherein the covering 314 has friction-reducing features 318 on the outer surface 316 of the covering 314. This embodiment also has slots 320 defined in the distal end that provide additional flexibility or reduced stiffness to the distal end of the shaft 312. The shaft 312 of this specific exemplary catheter 310 implementation has a smaller diameter at its distal end than the shaft 292 described above, either because the distal end is swaged or has a reduced diameter by comparison. Alternatively, the shaft 312 has a smaller diameter because a smaller diameter shaft is attached (in some embodiments via welding) to a distal end of a larger shaft 312. As with the catheter 290 above, the slots 320 in this embodiment can be substantially straight slots 320 as shown or alternatively configured in a partially or substantially spiral-like configuration. Alternatively, the slots 320 can have on any shape or configuration of an opening that can provide additional flexibility or reduced stiffness to the shaft 312. In accordance with one embodiment, the slots 320 can be configured such that the hypotube 312 is more flexible in the distal portion of the slots 320 in comparison to the proximal portion of the slots 320.

In use, an OTW catheter that is used for advancing past a narrowed length of a blood vessel is used in the following fashion. First, the guidewire is advanced into position past the stenosis. Typically, this guidewire is fairly flexible (less stiff). Next, the OTW catheter is advanced over the guidewire. Subsequently, the first guidewire is removed and replaced with a second, stiffer guidewire, which can be used to help advance a second interventional device past the lesion over the second guidewire.

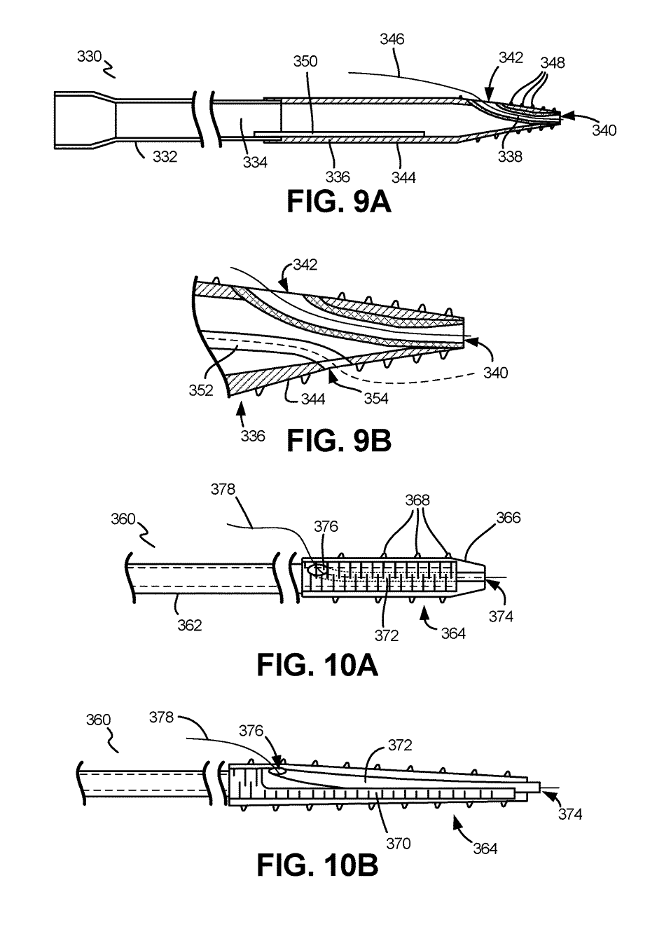

Alternatively, the catheter embodiments disclosed or contemplated herein can be rapid-exchange catheters. One such exemplary embodiment is depicted in FIG. 9A, which depicts a rapid-exchange catheter 330 having a shaft 332 with a lumen 334 defined therein and a polymeric tube 336 coupled to the distal end of the shaft 332. The tube 336 has a separate guidewire lumen 338 defined therein that has a distal opening 340 at the distal end of the tube 336 and a proximal opening 342 defined along the outer surface 344 of the tube 336 such that the lumen 338 is configured to receive a guidewire 346 as shown. The tube 336 in this embodiment has friction-reducing features 348 disposed on the outer surface 344 of the tube 336. In certain implementations, the tube 336 is tapered as shown. According to some embodiments, the tube 336 has a support rod or wire 350 disposed in the tube 336 that provides additional structural support and/or stiffness to the tube 336.

FIG. 9B depicts an expanded view of the distal end of the tube 336 of FIG. 9A. In this alternative implementation, the tube 336 has a secondary lumen 352 with an opening 354 defined in the outer surface 344 and extending proximally to the lumen 334 of the shaft 332 of the catheter 330. The secondary lumen 352 can be used for fluid injection or for insertion or removal of another guidewire.

Yet another implementation of a rapid-exchange catheter is shown in FIGS. 10A and 10B, in which the catheter 360 has a metal shaft 362 (also referred to as a hypotube 362) with a polymeric covering 364 disposed on the distal end of the shaft 362. The polymeric covering 364 has friction-reducing features 368 disposed on the outer surface 366 of the covering 364. Further, the distal end of the shaft 362 may also have a length 370 with a reduced diameter, wherein the polymeric covering 364 is positioned over the reduced diameter length 370. In the embodiment as shown, the reduced diameter length 370 is created by a portion of the hypotube 362 being cut away, thereby increasing the flexibility and/or reducing the diameter of the hypotube 362. Alternatively, the reduced diameter length 370 can have any known reduced diameter configuration. In addition, in certain embodiments, the polymeric covering 364 has a separate guidewire lumen 372 defined therein that has a distal opening 374 at the distal end of the tube covering 364 and a proximal opening 376 defined along the outer surface 366 of the covering 364 such that the lumen 372 is configured to receive a guidewire 378 as shown.

In use, a rapid-exchange catheter that is used for advancing past a narrowed length of a blood vessel is used in the following fashion. First, the fairly flexible guidewire is advanced into position past the stenosis. Next, the rapid-exchange catheter is advanced over the guidewire by positioning the guidewire through the guidewire lumen (such as guidewire lumen 338 discussed above). Subsequently, the first guidewire is removed and replaced with a second, stiffer guidewire, which is inserted through the catheter via the secondary lumen (such as secondary lumen 352 discussed above). The second guidewire can ultimately be used to help advance a second interventional device past the lesion over the second guidewire.

Another embodiment of a catheter 390 is depicted in FIG. 11A, in which the catheter 390 has a shaft 392 made entirely of metal and a distal end with slots 394 defined therein to add flexibility to the catheter 390 for navigation of the vasculature. The slots 394 can take on any configuration or have any of the features as described with respect to the slots 300, 320 discussed above. Further, the outer surface 396 of the shaft 392 has friction-reducing features 398 at the distal end of the shaft 392. In one implementation, the shaft 392 has a lubricious coating (not shown) of any known material (such as Teflon, for example) disposed around the shaft 392. In one embodiment, the pattern of slots 394 is depicted in FIG. 11B, while another embodiment is depicted in FIG. 11C. The catheter 390 can also have a polymeric tube 400 (also referred to herein as an “inner lumen liner”) as shown in FIG. 11D that can be positioned in the inner lumen 402 of the shaft 392 to assist with passage of any guidewires or other devices through the lumen 402. In one embodiment, the polymeric tube 400 has two layers: an inner layer 404 and an outer layer 406. According to certain implementations, the outer layer 406 is made of a polymeric or metal material, while the inner layer 404 is made of a polymeric material that aids in the passage of any device being urged through the tube 400. For example, in one embodiment, the inner layer 404 is made of a lubricious material.

According to one embodiment, any friction-reducing features—including, for example, coils, including in a braided configuration—can be positioned on any length of the outer surface 396 of the shaft 392. Further, as discussed above, any coils or tip disposed on the shaft 392 can extend past the distal end and have a folded configuration in which the distal end of the coil or tip is positioned in the inner lumen of the catheter 390 in an invaginating manner.

It should be noted that, in any of the embodiments disclosed or contemplated herein in which different components or layers are bonded together—such as, for example, a coil bonded to the outer surface of a hypotube—an adhesive (also referred to as an “adhesive layer” or “bonding layer”) may be used to fully or partially bond those features together. Thus, in some embodiments, the adhesive layer could be used to bond any friction-reducing feature to a shaft of a catheter or any other component. This adhesive can be any known adhesive or polymer that can attach or bond any such two components together. For example, an adhesive polymeric layer such as LLDPE or cyanacrylate can be used to bond a metallic braid to a metallic hypotube or polymeric tube. Alternatively, rather than an adhesive layer, a welding or soldering type process may be used to attach a metallic surface component to a metallic tube (such as, for example, attaching a metallic braid to a metallic hypotube using a laser welding process).

Although the present invention has been described with reference to preferred embodiments, persons skilled in the art will recognize that changes may be made in form and detail without departing from the spirit and scope of the invention.

What is claimed is:

1. A catheter for advancing past a vascular stenosis, the catheter comprising:
(a) a catheter body comprising a lumen defined therein; and
(b) at least one friction-reducing feature associated with the catheter body.

2. The catheter of claim 1, wherein the at least one friction reducing feature is disposed on an outer surface of the catheter body.

3. The catheter of claim 1, further comprising a tip coupled to a distal end of the catheter body, wherein the at least one friction reducing feature is disposed on an outer surface of the tip.

4. The catheter of claim 1, wherein the at least one friction-reducing feature comprises a plurality of projections.

5. The catheter of claim 4, wherein the plurality of projections comprise bumps, quadrangular projections, or triangular projections.

6. The catheter of claim 1, wherein the at least one friction-reducing feature comprises a plurality of openings or dimples defined in the outer surface of the catheter body.

7. The catheter of claim 1, wherein the at least one friction-reducing feature comprises a plurality of grooves, nubs, ribs, or textured features.

8. The catheter of claim 1, wherein the at least one friction-reducing feature comprises at least one wire formed into a coil configuration or a braided configuration.

9. The catheter of claim 1, wherein the at least one friction-reducing feature comprises at least one offset projection.

10. The catheter of claim 1, further comprising an exterior layer disposed over an outer surface of the catheter body and the at least one friction-reducing feature.

11. The catheter of claim 1, further comprising a lubricious coating disposed over at least an outer surface of the catheter body.

12. The catheter of claim 1, wherein the catheter body comprises a length having a reduced diameter, wherein the at least one friction reducing feature is disposed on an outer surface of the length having the reduced diameter.

13. The catheter of claim 12, further comprising a tip positioned over the length having the reduced diameter, wherein the at least one friction reducing feature is disposed on an outer surface of the tip.

14. The catheter of claim 1, wherein the catheter shaft is a hypotube.

15. The catheter of claim 14, wherein the catheter shaft further comprises at least two slots defined in the a distal end of the catheter shaft.

16. The catheter of claim 15, wherein the at least two slots comprise a straight configuration or a spiral-like configuration.

17. The catheter of claim 1, wherein the catheter shaft comprises a first length comprising a first diameter, a second length comprising a second diameter, and a transition portion disposed between the first and second lengths.

18. The catheter of claim 1, wherein the catheter shaft comprises a first layer and a second layer.

19. The catheter of claim 1, wherein the catheter comprises an over-the-wire catheter or a rapid-exchange catheter.

20. The catheter of claim 1, further comprising a rotation mechanism associated with a distal end of the catheter body, wherein the rotation mechanism is configured to rotate when actuated.

21. A method of advancing a catheter past a vascular stenosis, the method comprising:
positioning a catheter into a blood vessel, the catheter comprising:
(a) a catheter body comprising a lumen defined therein; and
(b) at least one friction-reducing feature associated with the catheter body;
advancing the catheter past the vascular stenosis, wherein the at least one friction reducing feature reduces friction between the vascular stenosis and the catheter.

22. The method of claim 21, further comprising rotating a distal end of the catheter body, wherein the rotation reduces friction between the vascular stenosis and the catheter.

23. The method of claim 21, further comprising moving the distal end of the catheter body in a lateral direction, wherein the movement reduces friction between the vascular stenosis and the catheter.
